(19) United States
(12) Patent Application Publication
(10) Pub. No.: US 2019/0142364 A1
(43) Pub. Date: May 16, 2019

(54) METHOD FOR MEASURING CARDIAC OUTPUT BY ULTRASOUND

(71) Applicants: CENTRE HOSPITALIER UNIVERSITAIRE D'AMIENS-PICARDIE, Amiens (FR); UNIVERSITE DE PICARDIE JULES VERNE, Amiens Cedex (FR); INSTITUT NATIONAL DE LA SANTE ET DE LA RECHERCHE MEDICALE (INSERM), Paris (FR)

(72) Inventors: Michel SLAMA, Amiens (FR); Julien MAIZEL, Amiens (FR); Christophe BEYLS, Amiens (FR); Roger BOUZERAR, Abbeville (FR); Olivier BALEDENT, Corbie (FR)

(73) Assignees: CENTRE HOSPITALIER UNIVERSITAIRE D'AMIENS-PICARDIE, Amiens (FR); UNIVERSITE DE PICARDIE JULES VERNE, Amiens Cedex (FR); INSTITUT NATIONAL DE LA SANTE ET DE LA RECHERCHE MEDICALE (INSERM), Paris (FR)

(21) Appl. No.: 16/308,213

(22) PCT Filed: Jun. 6, 2017
(86) PCT No.: PCT/EP2017/063718

§ 371 Date: Dec. 7, 2018

(30) Foreign Application Priority Data
Jun. 10, 2016 (FR) 1655360

(51) Int. Cl. A61B 8/06; A61B 8/08; A61B 8/00; A61K 49/22

(52) U.S. Cl. CPC A61B 8/065; A61B 8/481; A61K 49/223; A61B 8/5223; A61B 8/463

(57) ABSTRACT

Method for measuring cardiac output using an ultrasound apparatus, wherein: a plurality of images of an area of the heart where a contrast agent has been injected is acquired by the ultrasound apparatus, at different times; values of the intensity of the area are extracted from the images; and the cardiac output is calculated from the value given to at least one parameter (k) of a reference function, adjusted so that said function best describes the changes in the intensity values measured.

The present invention concerns the measurement of cardiac output.

Cardiac output represents the quantity of blood ejected by the heart per minute and is the product of the quantity of blood ejected by the heart at each contraction, or the systolic ejection volume, times the heart rate. In people in intensive care or under anesthesia with hemodynamic instability and in a state of shock, this parameter must be monitored closely. Various methods permit measuring cardiac output.

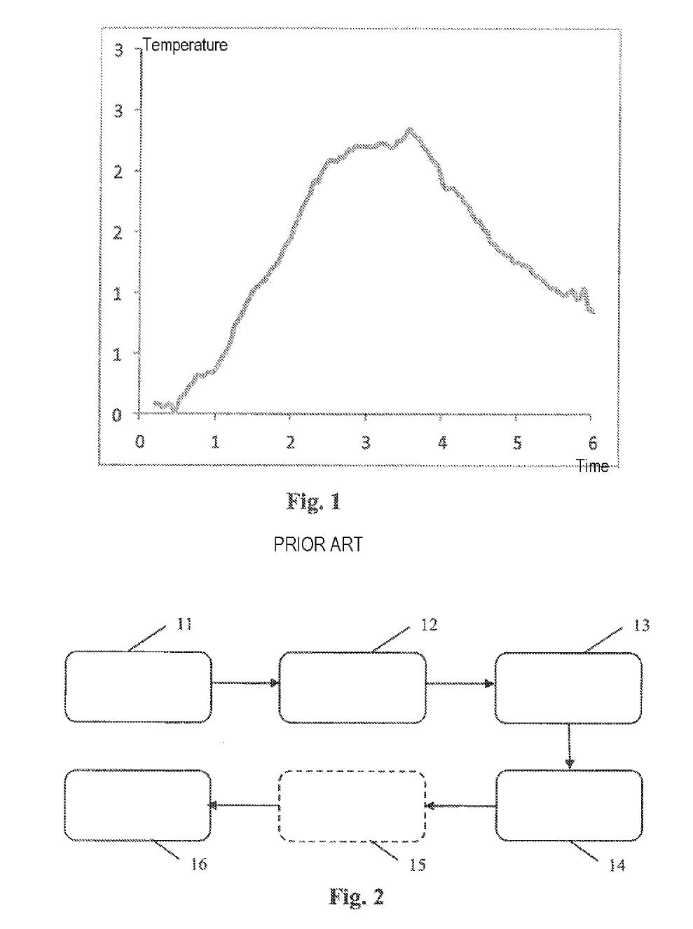

One of the main techniques is the method called “thermodilution”. A so-called “Swan-Ganz” catheter is introduced through a large central vein and pushed into the right heart then into the pulmonary artery. This catheter has outlet openings at the right atrium and pulmonary artery. Low inertia thermistors are present in the catheter to measure small variations in blood temperature. A cold liquid bolus is injected by an opening into the right atrium, cooling the blood a few tenths of a degree. A temperature variation is recorded at the end of the catheter, and has the behavior shown in FIG. 1. Knowledge of the temperature variation permits calculating cardiac output. A large number of animal and human studies have validated this measurement technique and have made it the standard technique for measuring cardiac output, in particular in intensive care patients. A variant called transpulmonary thermodilution has also been developed, allowing and collecting temperature variations in a large artery.

These thermodilution techniques are invasive, since it is necessary to position one or more intravascular catheters. The complications associated with such procedures are well known, especially mechanical ones related to the difficulties of positioning catheters, such as the onset of a hematoma, pneumothorax or hemopericardium, as well as thromboembolic or infectious complications.

Cardiac output can be also measured by Doppler echocardiography (DE).

DE precisely calculates the flow rates within the heart. The systolic ejection volume can be calculated from two measurements: the diameter of the aorta D, on the one hand, and the aortic velocity time integral VTIao recorded in pulsed Doppler. During contraction of the left ventricle, a certain quantity of blood is ejected into the aorta at a given rate. This volume of blood flows into the aorta for a distance that can be calculated if the mean velocity and duration of ejection is known. DE permits this mean velocity to be measured and the ejection duration to be known. It is therefore possible to calculate the distance traveled by a column of blood in the aorta, treated as a cylinder. Cardiac output CO is obtained by multiplying this volume by the heart rate according to the formula CO=(πD²/4)×VTIao×HR. This technique is noninvasive, relatively simple to implement and the output measured is well correlated to the measure of output by thermodilution. However, this technique cannot be automated and is sometimes difficult to do in intensive care transthoracically because the patient's echogenicity may be poor, i.e., it is difficult to even see the cardiac structures. Moreover, since the output is obtained from a measurement of the diameter of the aorta squared, any error, however small, at this level can lead to major errors in calculating output.

In the case where the heart cannot be visualized by transthoracic echocardiography, it is possible to use a catheter that is placed in the esophagus. This technique is called transesophageal echocardiography. In the same way as by transthoracic approach, it is possible to measure the aorta diameter and flow to calculate cardiac output.

These echocardiography techniques cannot be automated and are operator-dependent. Moreover, certain diseases distort the measurement, such as an aortic valve anomaly or the existence of an obstruction at the outlet of the left ventricle.

International application WO 95/29705 describes a method to measure cardiac output, using ultrasound images and intravenous injection of a contrast medium comprising microbubbles. Cardiac output is calculated by integrating a relation between time and the video-density of the images obtained during the transit of the microbubbles as a function of the volume injected.

During an echocardiogram, it is common to have to look for a pathological hole between the right part of the heart and its left part, called patent foramen ovale, showing an intraatrial or intraventricular communication. To do this, a mixture of 90% saline serum and 10% air is used, done by using two syringes and pushing the mixture from one syringe to the other in order to obtain a solute with a multitude of microbubbles in suspension. This mixture, injected by a vein, intensely reflects ultrasound, creating a real contrast medium visible in ultrasound when it reaches the heart. If the product passes from the right part of the heart to the left part, there is a hole in the heart. This contrast disappears gradually. In the absence of a hole, the solution is diluted in the blood and does not go through the pulmonary capillaries.

Many scientific articles teach the measurement of certain cardiac parameters by injection of a contrast medium comprising microbubbles, by using images acquired by echocardiography when the product is diluted.

The article by Mehta et al. “Validation of a novel method for cardiac output estimation by CT coronography angiography” describes the correlation between cardiac output and attenuation of contrast in the aortic root after injection of microbubbles. The change in product concentration over time is determined by using regions of interest in the acquired images.

The article by Yun et al “Usefulness of ultrasound contrast media for cardiac output measurement with echocardiography”, Korean Journal of Veterinary Research, 2015, 55(1): pages 47-52, uses the attribution of scores to segments of the region analyzed after injection of a contrast medium to improve the visualization of regions of the heart and uses a Simpson method to calculate cardiac output.

The article by Jansen et al “Novel ultrasound contrast agent dilution method for the assessment of ventricular ejection fraction”, European Journal of Echocardiography, 2008, vol 9, pages 489-493, describes the use of dilution indicator curves after injection of microbubbles to measure the ventricular ejection fraction, corresponding to the volume of blood ejected.

The article by Choi et al “Estimation of cardiac output and pulmonary vascular resistance by contrast echocardiography transit time measurement: a prospective pilot study”, published in Cardiovascular Ultrasound, 2014, 12: 44, showed a correlation between transit time of the contrast medium and cardiac output, by using the transit time of the microbubbles between the right and left sides of the heart.

The article by Mischi et al “Videodensitometric methods for cardiac output measurements”, published in EURASIP Journal on Applied Signal Processing 2003: 5, pages 479 to 489, describes an experimental method for measuring cardiac output done in vitro, using a plastic bag to model the measurement region, a SonoVue™ contrast medium, being a stabilized gas and having a somewhat long life cycle, and a theoretical dilution model based on an infinite nondeformable cylinder with no recirculation. This article puts forward a statistical type algorithm for mapping dilution curves.

There is a need to further improve methods for measuring cardiac output, in order to have a less invasive, automated and reliable technique.

The invention aims to meet this need and does so, according to one of its aspects, via a method for measuring cardiac output using an ultrasound machine, method in which:
a plurality of images of a region of the heart where a contrast medium was injected is acquired by the ultrasound machine, at different times,
ultrasound signal intensity values of the region are extracted from the images, and
cardiac output is calculated from the value given to at least one reference function parameter, adjusted so that said function best describes the evolution of the intensity values measured.

“Ultrasound signal intensity of the region” means information representing the mean gray level of the pixels of the image in the region.

The invention permits a less invasive cardiac output measurement to be obtained, which can be easily automated and reproduced. The measurements may be reproduced since they are not dependent on an operator to do the measurements. The method according to the invention may be used during anesthesia of a patient at risk of instability. In intensive care, it may be used in all hemodynamically unstable patients.

In the same way that temperature variation during thermodilution allows measuring cardiac output, the invention exploits the fact that the variation and rate of disappearance of the contrast medium is correlated with cardiac output. The recording of the ultrasound signal related to the passage of microbubbles in the region of the heart observed therefore obeys physical laws which contain information on the flow rate of the blood transporting the microbubbles.

Thanks to the invention, it is possible to obtain the cardiac output measurement by automatically analyzing the rate of disappearance of the contrast medium. The contrast medium is preferably made up of a serum filled with air microbubbles. Such a contrast medium can be very easily generated and entirely resorbed in the body after the measurement.

The method for measuring cardiac output may therefore be preceded by the step of injecting air microbubbles as a contrast medium. This injection can take place automatically, at regular intervals. After each injection, cardiac output can also be measured automatically. An alarm may be generated if an abnormal cardiac output value is detected.

This method may also be implemented in animals, with sacrifice of the animal if necessary.

The method according to the invention is suited to be implemented in real time, allowing the medical team to react more quickly according to the calculated cardiac output value. It may also be implemented automatically at regular intervals to monitor the patient's status.

The invention may also be implemented by transthoracic or transesophageal echocardiography.

The contrast medium is introduced through the right atrium by means of a venous access. This access may be a jugular vein, located in the neck, or the femoral vein where contrast increases more slowly, or a peripheral vein.

Observation of the contrast and measurements of the ultrasound signal intensity are done at the right atrium and/or right ventricle. These heart chambers deform with systole and diastole. The blood leaves these chambers through valves that open and close with the heart rate.

Creation of the Contrast Medium

The invention also relates, according to another one of its aspects, to a device for creating air microbubbles as a contrast medium, notably for implementing the method for measuring cardiac output according to the invention such as defined above, the device comprising:
a syringe, connected to a valve,
a mechanism to automatically activate the syringe plunger several times in a row, for example between five and twenty times, by pulling the plunger with the valve closed to create a vacuum inside the syringe, then by releasing the traction in order to break the vacuum.

The use of such a device to create the contrast medium allows obtaining a standard and reproducible contrast in the ultrasound image, which is not operator-dependent.

The device for creating a contrast medium according to the invention may be adapted to an existing ultrasound machine or be part of an independent system.

The contrast medium is preferably a sterile mixture of serum and air, notably containing between 5 ml and 10 ml of saline serum and between 0.5 ml and 1 ml of air, for example 4 ml of serum and 0.5 ml of air.

The valve can be controlled automatically by opening once the vacuum and repressurization cycle is finished, for the injection of the serum filled with air microbubbles.

Once the contrast medium is injected, the air microbubbles formed by the mixture do not pass from the right ventricle to the left ventricle, being stopped by the lungs. Microbubbles have a short life cycle, making the presence of the contrast medium in the region of the heart where it is injected brief.

Analysis of the Images

The plurality of images of a region of the heart where the contrast medium was injected is acquired by the ultrasound machine, at different times, preferably while noting the time elapsed between these images.

The images may be recorded in DICOM (digital imaging and communications in medicine) format, the standard for computer management of medical imaging data. As a variant, the images are recorded in any possible export format, for example video.

An intermediate image of the MIP type, for “maximum intensity projection”, may be created to evaluate the extension of the contrast in the image.

The region of interest of the region of the heart may be determined manually or automatically, being carried over to all of the images acquired over time.

The contour of the region of interest is, for example, circular, oval, polygonal, or other, and follows the morphology of the right chambers, for example.

The signal intensity is obtained, for example, by averaging the gray levels of the image pixels inside this contour.

The change in contrast over time from the intensity values of each image, notably in the region of interest, is advantageously recorded.

Calculation of Cardiac Output:

A signal representing the increase and decrease, over time, of the intensity values of the area observed, extracted from the images, may be obtained. This signal may be adjusted by a reference curve, product of a decreasing exponential and a power of time: y=a·t^{b}·e^{-kt}·(k>0; b>or=to 0).

The signal envelope is advantageously similar to the signal obtained by the known technique of thermodilution. A formula identical to the one used for thermodilution, applied to this signal in its entirety, may then be used to calculate cardiac output.

In one variant, a signal is obtained only representing the decrease over time of the intensity values of the region, extracted from images. These signals show the evolution of the contrast medium injected into the region of the heart being studied.

The reference function to determine the quantity of contrast medium Q(t) present in a chamber where it is flowing, by weight assessment, may be expressed as follows:

dQ(t)=−r·c(t)·dt=−r·Q(t)/V·dt

which leads to

Q(t)=Q₀·e^{-kt}

where k=r/V represents the ratio between the output flow rate r and V the volume of the area observed. This ratio k is the parameter of decrease of the reference function.

Cardiac output is calculated from the value given to at least one decrease parameter k, adjusted so that said function best describes the evolution of the intensity values measured.

In the case where the signal representing only contrast decrease is recorded, the maximum Sₘₐₓ of the signal may be defined, as well as the time Tₘₐₓ corresponding to it. The signal may be normalized by the maximum Sₘₐₓ and repositioned on the x-axis with respect to the corresponding time Tₘₐₓ. A low pass filter may be applied onto the contrast decrease signal curve, in order to smooth the curve.

Relationship between the Parameter k and the Size of the Area Studied

Decrease parameter k is directly linked to the volume/area of the chamber observed. If this volume is very large, there is an underestimate of the parameter k value measured and therefore the cardiac output calculated.

For patients with chambers with abnormally high volume, especially the atrium, the value of decrease parameter k may be corrected by multiplying it by the ratio of the chamber area to the mean area normally observed for this chamber.

The mean area normally observed for the chambers may be obtained by taking the mean of the areas obtained for a certain number of so-called standard patients according to ultrasound recommendations.

The method may thus include the step consisting of determining the area of the region of interest used for measuring the ultrasound signal intensity and calculating the cardiac output by considering this area, either applying a correction to reduce the measured values to values comparable to those obtained for a reference area region, or by using different reference curves as a function of the area of the region observed.

Computer Program Product

The invention also relates, according to another of its aspects, to a computer program product for the implementation of the method for measuring cardiac output using an ultrasound machine such as defined previously, the computer program product including code instructions which, when executed by a processor, do the following:
a plurality of images of a region of the heart where a contrast medium was injected is acquired by the ultrasound machine, at different times,
signal intensity values of the region are extracted from the images, and
cardiac output is calculated from the value given to at least one reference function parameter, adjusted so that said function best describes the evolution of the intensity values measured.

The characteristics stated above for the method according to the invention apply to the computer program product.

The computer program product according to the invention can be integrated into an ultrasound machine, being notably recorded on a microprocessor card integrated into the echocardiography machine. In one variant, the computer program product is integrated into an external system.

### DETAILED DESCRIPTION

The invention may be better understood upon reading the detailed description that follows, of non-limiting examples of embodiment thereof, and on examination of the attached drawing, in which:

FIG. 1 shows the change over time of a temperature signal obtained by the thermodilution method of the prior art,

FIG. 2 illustrates the steps for implementing an example of the method according to the invention,

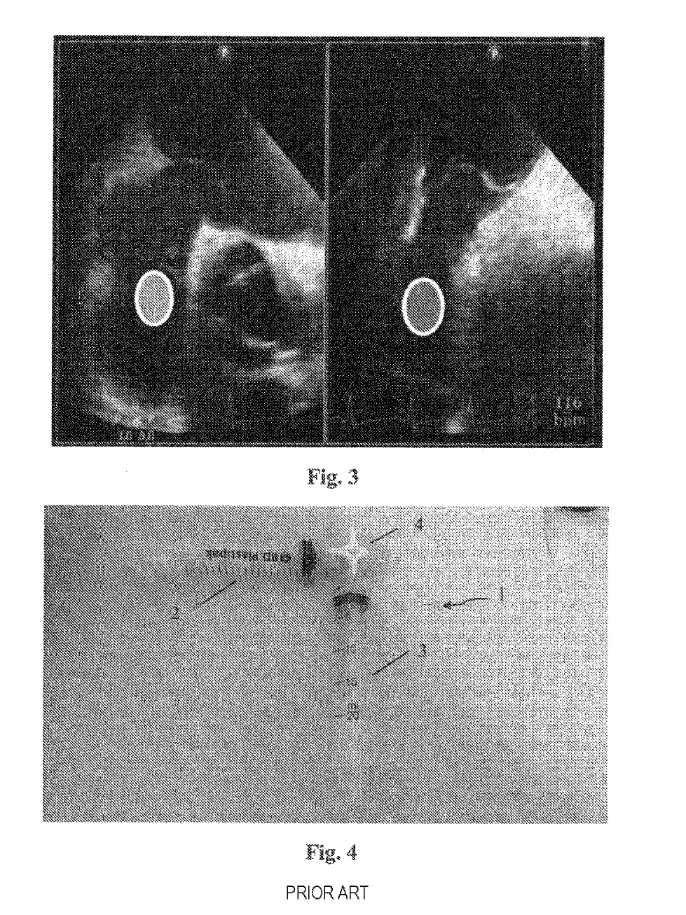

FIG. 3 shows images of transesophageal echocardiography used to implement the invention, showing the contour of the region of interest,

FIG. 4 shows a device according to the prior art for creating the contrast medium,

FIG. 5 shows a device according to the invention for creating the contrast medium,

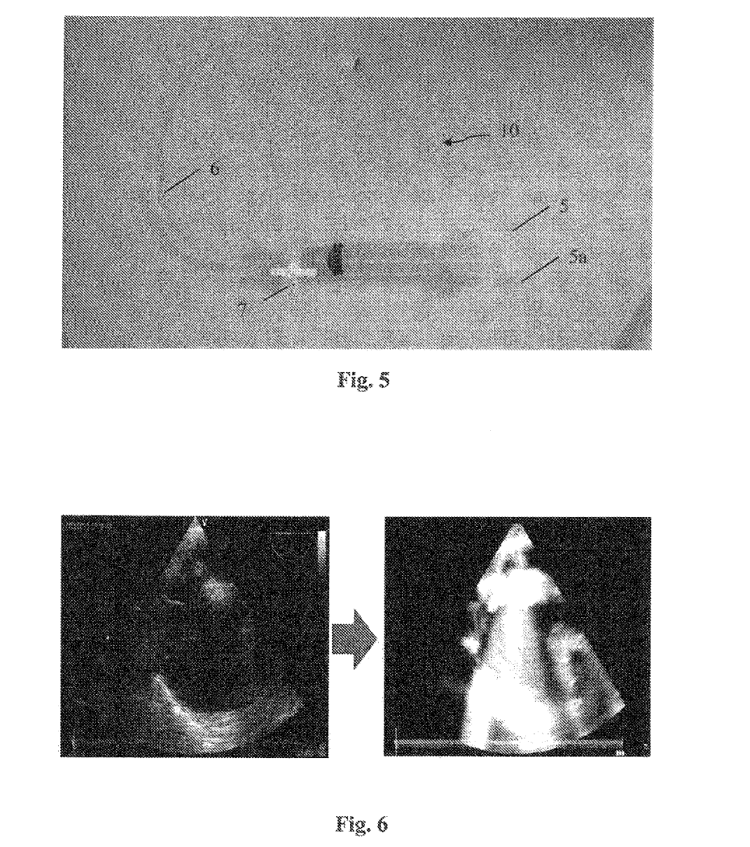

FIG. 6 illustrates the appearance of contrast related to the use of the contrast medium,

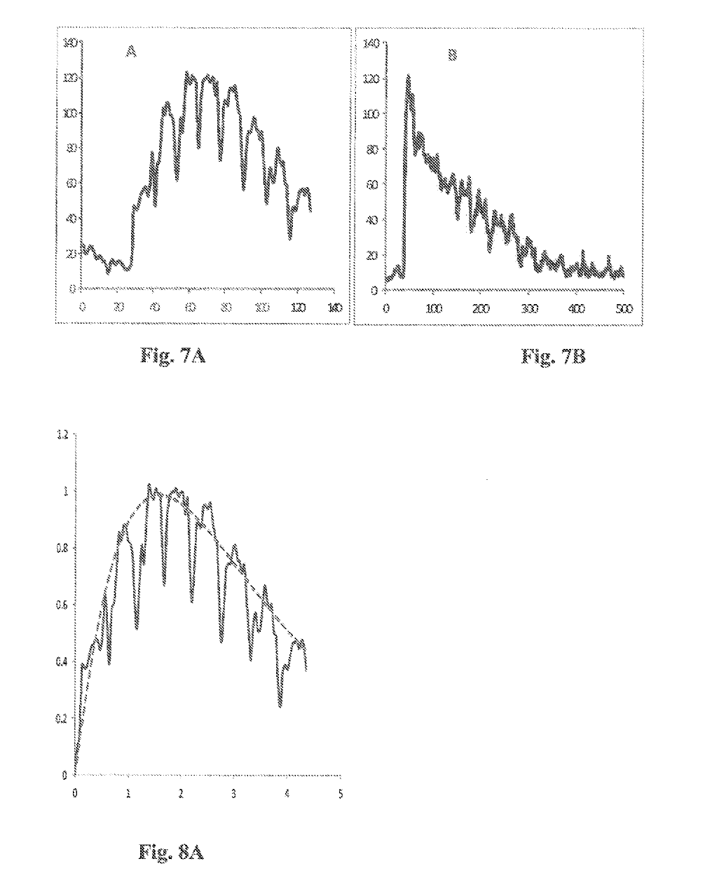

FIGS. 7A and 7B show timing diagrams of the intensity value signals obtained by applying the method according to the invention,

FIG. 8A shows an example of the contrast evolution signal obtained according to the invention and adjusted,

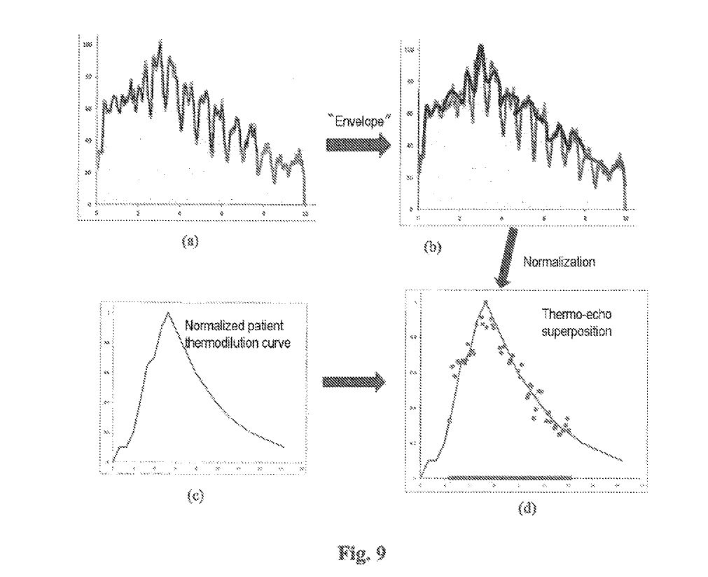

FIGS. 9a to 9d illustrate another example of the comparison of signals obtained according to the invention and according to the prior art,

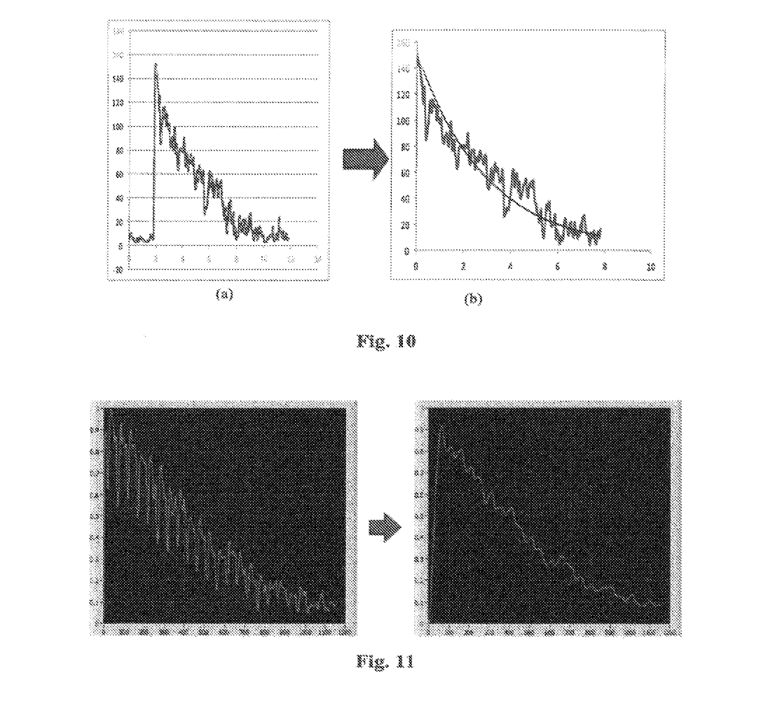

FIGS. 10a and 10b show the decrease signal of the intensity values obtained by applying the method according to the invention,

FIG. 11 shows another example of the decrease signal, after normalization, and before and after filtering,

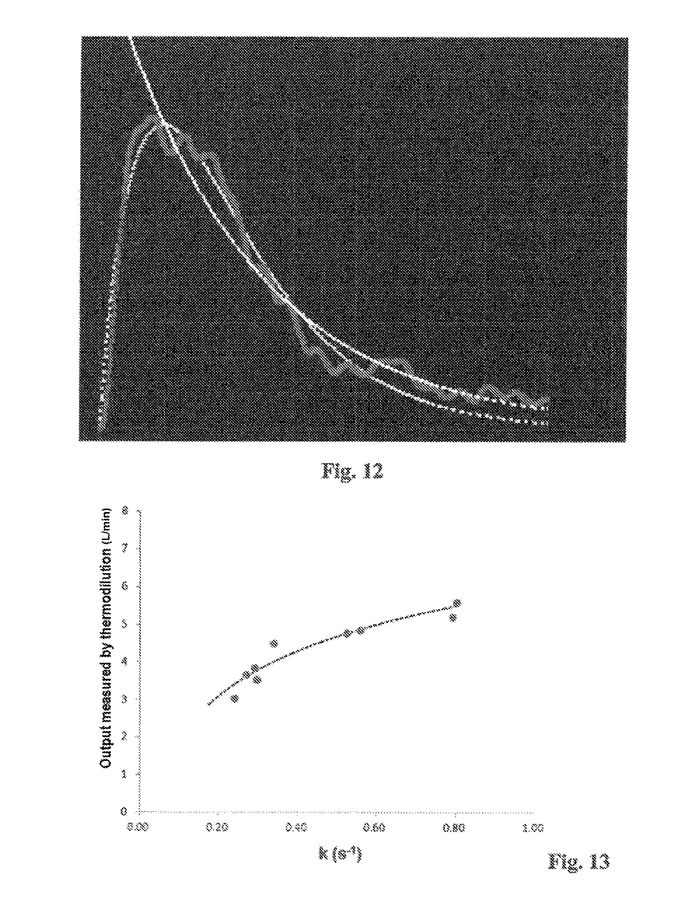

FIG. 12 illustrates the fitting of the reference function to make it correspond to the decrease curve of FIG. 11 after filtering,

FIG. 13 shows the correlation between cardiac output measured by thermodilution and decrease parameter k,

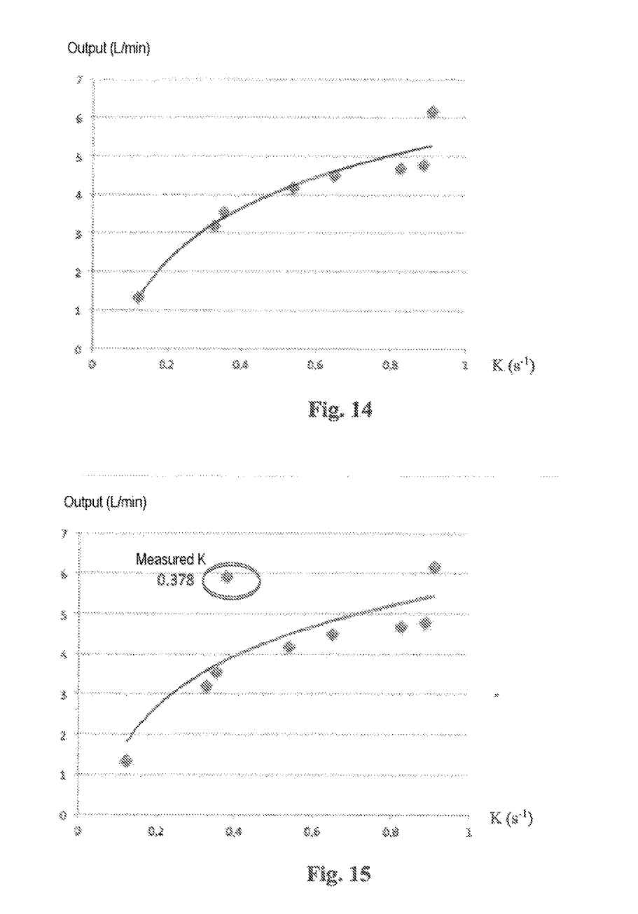

FIG. 14 is a curve showing cardiac output calculated according to decrease parameter k, for patients with a standard right atrium area,

FIG. 15 shows the inclusion in the curve of FIG. 14 of a patient with a dilated right atrium (area 28 cm²),

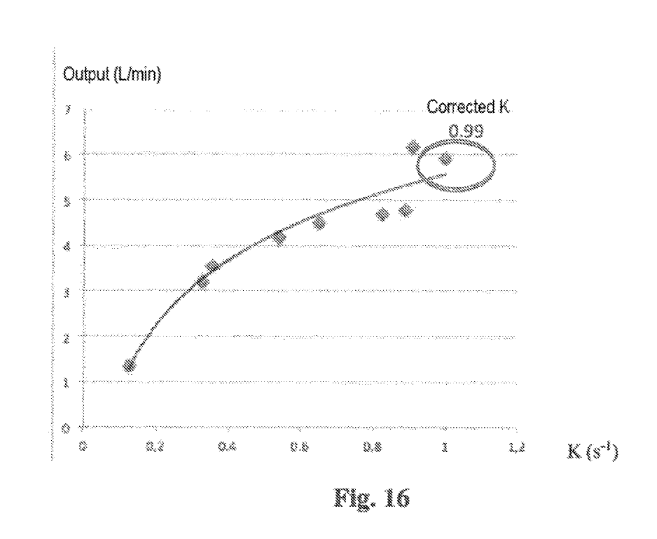

FIG. 16 shows the curve of FIG. 15 with correction of decrease parameter k.

Different steps are shown in FIG. 2 for implementing the method for measuring cardiac output of a heart according to the invention.

During a first step 11, the patient is prepared for an echocardiogram, to measure their cardiac output, among other parameters.

In one example of embodiment of the invention, a transesophageal echocardiography probe is used to obtain an image of the right atrium RA, as shown in FIG. 3.

One or more syringes of serum, for example five, containing 4 ml of saline serum and 0.5 ml of air, are prepared beforehand. The invention is not limited to a particular type of contrast medium, although a serum/air mixture is widely preferred.

The contrast medium can be made by using the device as illustrated in FIG. 4, including two syringes 2 and 3, connected by a three-way valve 4. The serum contained in the first syringe 2 is propelled toward the second empty syringe 3, then reinjected into the first syringe 2 in order to obtain a homogeneous solution containing air microbubbles.

As a variant, a device 10 according to the invention such as shown in FIG. 5 is used, comprising a single syringe 5, connected to a valve 7 and an extender 6, designed to be connected to the injection catheter. Plunger 5a of syringe 5 may be activated automatically several times in a row, by a mechanism that is not shown, for example 10 times, to form the air microbubbles. At each pull of plunger 5a, a vacuum is created in the syringe, then plunger 5a is released to break the vacuum.

Then valve 7 is opened, and the serum containing the air microbubbles is injected.

Step 12 in FIG. 2 corresponds to the injection of the contrast medium via a catheter into a jugular vein or by the injection route located on the Desilet introducer of the Swan-Ganz catheter.

During a step 13, a recording of a plurality of images is made from the start of injection to the complete disappearance of the contrast in ultrasound. A contrast test example according to the invention is shown in FIG. 6.

Each image is analyzed in terms of signal contrast by an image analysis. In the example considered in a step 14, the region of interest that is defined by an oval contour in FIG. 3, is placed in the ultrasound images, according to the experimental conditions, notably the injection site.

The ultrasound signal intensity is the mean of the gray levels of the pixels located in the region of interest.

The evolution of signal A obtained when the injection is done far from the right atrium, notably in the femoral vein, is shown in FIG. 7A. An increase and decrease of the signal intensity are observed in the region of interest, as a function of time. This signal A, which corresponds to the arrival of the contrast medium then to its departure, is modulated by the heartbeats. The envelope of signal A is similar to the signal obtained by the thermodilution technique shown in FIG. 1. Cardiac output is then calculated during step 16.

In order to make the comparison with the thermodilution measurement method, at least three syringes of 5 to 10 ml of microbubble solution were used, and the experiment was repeated with each of these syringes. The contents of each syringe are injected into the right atrium using a catheter. The cardiac output by thermodilution is obtained with the temperature decrease curve, as previously explained.

The A and thermodilution signals can be adjusted by a reference curve, produced by a decreasing exponential and a power of time, as previously described. One fitting example is shown in FIG. 8A by the dotted curve.

FIG. 9 shows a type of signal A, from which is extracted the points of the envelope that are superposed on the corresponding thermodilution curve. The superposition is noteworthy. It confirms the similarity between the curves obtained by the prior art and by the invention and shows that the dispersion of a contrast medium can serve as the basis of the cardiac output measurement.

In a variant or in combination, a second signal B, shown in FIG. 7B, is obtained, where only the decrease in contrast is recorded. In this case, the decrease is adjusted by an exponential equation, as described previously. An example of signal B extracted from a patient's ultrasound images is shown in curve (a) of FIG. 10. The origin of the curve on the x-axis is shifted, with fitting by the reference function, after having determined the maximum signal Sₘₐₓ and corresponding time Tₘₐₓ, as visible in curve (b) of FIG. 10. Curve fitting by a decreasing exponential function of decrease parameter k is satisfactory.

In the example considered, during step 15, a low-pass filtering of contrast decrease signal B is done and leads to curve (b) of FIG. 11.

As previously described, fitting by the reference function of signal B is advantageously done, from curve (b) of FIG. 11 in the example considered, as illustrated in FIG. 12. The best fit is sought by working on the value of parameter k, so that the reference function best describes the evolution of the intensity values measured.

Parameter k can therefore be extracted when testing each patient, optionally averaged over several contrast tests conducted, as well as the quality of the fitting.

The correlation between cardiac output and decrease parameter k is established from the values of parameter k whose flow rate measured by thermodilution is known, as shown in FIG. 13. These preliminary results indicate an excellent correlation between the flow rate measured by thermodilution and the parameter k measured according to the invention, with a p error less than 5.10⁻⁴.

The curves of FIGS. 14 to 16 show examples of cardiac output calculated as a function of decrease parameter k, according to the size of the patient's right atrium. FIG. 14 corresponds to patients with a right atrium area less than 18 cm². The value obtained for a patient with a right atrium with an area of 28 cm² was included in this curve of FIG. 15. Decrease parameter k is overestimated in this case. As previously described, as shown in FIG. 16, decrease parameter k is then advantageously corrected, for example by the ratio of the area of the region of interest observed to the mean area normally observed for this region.

The invention can be used in patients hospitalized in medical intensive care for shock and/or respiratory distress. Preferably, the measurement is made after stabilization of the hemodynamic status, i.e., they have not experienced blood pressure or heart rate variation, any therapeutic change or any change in the respirator setting, if applicable, for more than one hour. A transesophageal echography probe can be put in place and the measurements can be done. The probe can be left in place in order to monitor the patient's hemodynamic status.

The invention is not limited to the examples that have just been described.

It is possible to do the measurements transthoracically.

The injection of the contrast medium and extemporaneous preparation thereof prepared externally may be done automatically.

1. A method for measuring cardiac output using an ultrasound machine, comprising:
acquiring a plurality of images of a region of the heart where a contrast medium was injected by the ultrasound machine, at different times,
extracting signal intensity values of the region from the images, and
calculating cardiac output from the intensity values to at least one reference function parameter k, adjusted so that said function parameter describes an evolution of the intensity values measured.

2. The method as claimed in claim 1, further comprising obtaining a signal A representing the increase and decrease, over time, of the intensity values of the region, extracted from the images.

3. The method as claimed in claim 2, in which the signal A representing an increase and decrease, over time, of the intensity values is adjusted by a reference curve y=a·t^{b}·e^{-kt} product of a decreasing exponential and a power of time.

4. The method as claimed in claim 1, further comprising obtaining a signal B representing only the decrease, over time, of the intensity values of the region, extracted from the images.

5. The method as claimed in claim 4, further comprising calculating the cardiac output from the value given to a decrease parameter k, adjusted so that the reference function Q(t)=Q₀·e^{-kt} describes an evolution of the intensity values measured, where Q(t) is the quantity of contrast medium over time, k=r/V the ratio between the outlet flow rate r and V the volume of the region.

6. The method as claimed in claim 1, further comprising determining a region of interest used to calculate the intensity values, being reported on all the images acquired.

7. The method as claimed in claim 4, in which the maximum Sₘₐₓ of a decrease signal is defined, as well as the corresponding time Tₘₐₓ, the decrease signal being normalized by the maximum Sₘₐₓ and repositioned on the x-axis relative to the corresponding time Tₘₐₓ.

8. The method as claimed in claim 1, in which a low-pass filter is applied onto a contrast decrease signal curve.

9. The method as claimed in claim 1, in which, for patients with an abnormally large size of the region, the decrease parameter value k is corrected, by multiplying by the ratio of the area of the region to a normal mean area for this region.

10. The method as claimed in claimed in claim 1, in which the contrast medium is a mixture of serum and air.

11. A computer program product for the implementation of the method for measuring cardiac output using an ultrasound machine as claimed in claim 1, the computer program product comprising code instructions which, when executed by a processor, do the following:
acquire a plurality of images of a region of the heart where a contrast medium was injected by the ultrasound machine, at different times,
extract signal intensity values of the region from the images, and
calculate cardiac output from the values to at least one reference function parameter k, adjusted so that said function describes the evolution of the intensity values measured.

12. A device for creating air microbubbles as a contrast medium, designed to be injected into a region of the heart for the implementation of a method for measuring cardiac output as claimed in claim 1, the device comprising:
a unique syringe, connected to a valve and an extender designed to be connected to a catheter,
a mechanism configured to automatically activate plunger of the syringe several times in a row, a vacuum being generated within the syringe when the plunger is pulled back while the valve is closed, the plunger then being released to break the pressure.
